(19) United States
(12) Patent Application Publication
(10) Pub. No.: US 2026/0174652 A1
(43) Pub. Date: Jun. 25, 2026

(54) A COMPOSITION FOR IMPROVING EFFECTIVENESS OF DYE PRECURSORS FOR CHANGE OF COLOUR OF HAIR

(71) Applicant: CAVINKARE PVT.LTD., Chennai, Tamil Nadu (IN)

(72) Inventors: Shobana N S, Chennai (IN); Tara KANNAN, Chennai (IN); Sajini VADUKUMPULLY, Chennai (IN)

(73) Assignee: CAVINKARE PVT.LTD., Chennai, Tamil Nadu (IN)

(21) Appl. No.: 19/124,891

(22) PCT Filed: Oct. 18, 2023
(86) PCT No.: PCT/IN2023/050957

§ 371 Date: Apr. 28, 2025

(30) Foreign Application Priority Data
Oct. 27, 2022 (IN) 202241055393

(51) Int. Cl. A61K 8/41; A61K 8/49; A61Q 5/10

(52) U.S. Cl. CPC A61K 8/41; A61K 8/415; A61K 8/49; A61K 8/494; A61Q 5/10

(57) ABSTRACT

The present disclosure relates to a synergistic compositions for improving effectiveness of dye precursors for change of colour of hair comprising one or more phosphate esters in the ranges of 0.001 to 15 wt % and one or more carboxylic acid esters in the ranges of 0.001 to 15 wt % in a select ratio co-acting with an oxidative dye precursor in combination with oxidising agents in a cosmetically acceptable base, surprisingly imparts superior colour uptake with good grey hair coverage. Further, the present disclosure relates to a composition provided in different cosmetically acceptable forms including cream, shampoo, lotion, gel and foam with significantly reduced dye chemicals. The present disclosure relates to a composition that is safe, efficient, cost-effective, and requires a significantly reduced quantity of dye precursors.

### TECHNICAL FIELD

The present disclosure relates to a cosmetic composition. More specifically, the present disclosure relates to a composition for improving effectiveness of dye precursors for change of colour of hair comprising one or more phosphate esters in the ranges of 0.001 to 15 wt % and one or more carboxylic acid esters in the ranges of 0.001 to 15 wt % in a ratio ranging from 40:1 to 1:30 co-acting with an oxidative dye precursor in combination with oxidising agents in a cosmetically acceptable base, surprisingly imparts superior colour uptake and consequent reduction in the quantity of dye precursor in formulation. Further, the present disclosure relates to a composition provided in different cosmetically acceptable forms including cream, shampoo, lotion, gel, foam, paste, and mousse with significantly reduced dye chemicals. The present disclosure relates to a composition that is safe, efficient, cost-effective, and requires a significantly reduced quantity of dye precursors.

### BACKGROUND

Information provided in the “Background” section is included for setting the context and for understanding of the present invention. It is not an admission that any of the data included in the present inventive application is previous knowledge, is currently being claimed, or is part of any publication directly or implicitly referred to herein.

In the field of hair colouring, oxidative dyes/permanent dyes have gained more interest and importance. Generally, it consists of two components. One component contains the dye intermediates (primary dye intermediates and couplers) and the second component is the oxidant. Upon mixing of these two components in definite ratios, the colour is developed because of coupling reactions between primary dye intermediates and couplers in presence of the oxidant. The oxidative dye composition typically provides good wash and light fastness. With the use of suitable couplers, a broad spectrum of shades can be achieved. A large portion of the permanent hair coloring compositions, contains p-Phenylenediamine (PPD) or its derivatives as the primary dye intermediate, which is a known sensitizer. The compositions involving the use of oxidation dyes in a permanent hair colour system, the extent of occurrence of allergic reaction is high, especially with products containing p-Phenylenediamine (PPD). The oxidation of PPD leads to the formation of Bandrowski's base, which is capable of inducing skin sensitization and allergic contact dermatitis. Hence, a permanent hair coloring composition with significantly less quantities of PPD or its derivatives will cause lesser threat to the end user and thus have become the need of the hour. At the same time this technical advantage will accomplish additional benefits such as cost-effectiveness of the product and greater safety for the manufacturers.

References are invited for relevant prior arts that have been reported towards this goal are discussed hereunder.

US20190209441 A1 discloses a hair colouring or a bleaching kit, which contains a malodour suppressant that has the capability to block the odour produced by the alkalizing agent, such as ammonia. The hair colouring composition includes oxidative dye precursors, which includes primary intermediates and couplers. The kit comprises of 2 components, either one of these may comprise a gel-network system containing of multi-lamellar sheets and multi-lamellar vesicles both showing d-spacing. Agents like fatty alcohol, non-ionic surfactant and optional phosphate esters are involved in the gel network system. The specific combination of fatty alcohols, and non-ionic surfactants assists in reducing, or even eliminating, the ammonia odour, when combined with the specific malodour reducing compounds described hereinafter. The malodour suppressors include ingredients which comprise at least one functional group selected from the group consisting of keto, aldehyde, ether, ester, or hydroxyl group. Typically, two malodour suppressors are used, and it can be any of the components. In the detailed list of malodour suppressors, isoamyl laurate is listed as one. The concentration of these malodour suppressors can be present in the first composition in an amount of 0 to 70% by weight. The colouring composition comprise a total amount of oxidative dye precursors ranging up to 12%, alternatively from 0.1% to 10%, alternatively from 0.3% to 8%, alternatively from 0.5% to 6%, by total weight of the composition. This prior art discloses hair colouring and bleaching compositions with a reduced or no ammonia odour, however, the impact of malodour suppressors on dye intermediate usage levels is not discussed. Moreover, the patent does not discuss or indicate the impact of synergy between malodour suppressors and phosphate esters on the dye precursor concentration.

US20210177717A1 discloses a hair colouring formulation which is m-Aminophenol free. This composition is said to provide stable colour that does not change with time. This formulation comprises a mixture of dyes consisting of Methoxymethyl-p-Phenylenediamine, Hydroxyethyl-3,4-methylenedioxyaniline and 5-Amino-6-chloro-o-cresol, an alkaliser, and optionally other oxidative dyes provided that are not p-Phenylenediamine or p-Toluenediamine. The oxidative dyes present in the composition in a weight percentage ranging from 0.01 to 6% of the weight of the composition. Additionally, as a substitute for silicone such as coco-caprylates, iso-dodecane, C15-19 alkanes and isoamyl laurate are tried. Isoamyl laurate included in the composition in an amount ranging from 0.01 to 10% of the weight of the composition. The presence of isoamyl laurate not only gives the hair cosmetic properties (combability, shine) but also improves the fluidity of the mixture, making the mixture of dye and peroxide easier to apply. The advancement discloses m-Aminophenol free hair colouring composition and also conventional primary dye intermediates such as p-Phenylenediamine or p-Toluenediamine is not used. This prior art does not discloses dye intermediates reduction with the use of isoamyl laurate or phosphate esters.

WO2001062216A2 reveals a composition for conditioning keratin fibre, especially human hair. This patent also emphasizes on the use of a silicone-free composition, which is generally used for conditioning purpose due to few drawbacks such as high energy consumption during its synthesis and not being environmentally friendly. Hence in this prior art, at least one selected cosmetic carrier have been included, which is generally a cationic surfactant and at least one selected ester of 3-methyl-1-butanol in the cosmetic carrier. The ester of 3-methyl-1-butanol includes isoamyl laurate, isoamyl caprylate, isoamyl caprinate, isoamyl caprate, isoamyl myristate, isoamyl palmitate, isoamyl stearate, isoamyl oleate, isoamyl linolate, isoamyl linoleate, and isoamyl behenate and mixtures of these substances are particularly preferred. In the above patent, isoamyl laurate and other ester of 3-methyl-1-butanol are considered for conditioning property and commercially available hair colouring compositions are used to investigate the conditioning property. This prior art does not disclose dye reduction using carboxylic acid esters or phosphate esters.

US20030226217 A1 describes a composition suitable for dyeing and bleaching hair comprising a phosphate ester compound and an oxidizing agent. The phosphate ester compound preferably comprises a mixture of monoester phosphates of alkoxylated fatty alcohols containing from 12 to 22 carbon atoms with diester phosphates of non-alkoxylated fatty alcohols containing from 12 to 22 carbon atoms. They impart good rheologies and improve the bleaching and dyeing of hair. The colour uptake of the dyeing composition was improved significantly with incorporation of the phosphate ester compound. But the advancement does not disclose any impact on the usage of the dye precursors with the incorporation of phosphate ester compound. Moreover, carboxylic acid esters are not part of the composition.

A hair colouring composition or kit that exhibits superior colour uptake, which reduces the need for dye precursors, enhanced colour retention, uniform grey hair coverage, and good conditioning properties is urgently needed in the current scenario, where safety plays a crucial role. Additionally, this technological advantage would result in a significant reduction in the cost of the formulation, which will help the end users economically. Further, the present disclosure relates to the composition that is safe, efficient, cost-effective, and requiring significantly reduced quantity of dye precursors.

### Objectives

Primary object of the present disclosure is to provide a synergistic composition promoting uptake of hair colour in hair comprising reduced quantity of dye intermediate/precursors yet favouring superior colour delivery with good grey hair coverage.

An object of the present disclosure is to provide said synergistic composition for improving effectiveness of dye precursors to change of colour of hair comprising one or more phosphate esters in the ranges of 0.001 to 15 wt % and one or more carboxylic acid esters in the ranges of 0.001 to 15 wt % in a ratio ranging from 40:1 to 1:30 co-acting with an oxidative dye precursor in combination with oxidising agents in a cosmetically acceptable base.

Another object of the present disclosure is to provide said synergistic composition having significantly reduced quantity of oxidative dye precursors along with a synergistic combination of phosphate esters and carboxylic acid esters.

Yet another object of the present disclosure is to provide said synergistic composition promoting uptake of hair colour in hair with superior colour uptake, uniform grey hair coverage and good conditioning properties.

Further object of the present disclosure is to provide said composition which would utilize lesser quantity of dye precursors thereby reduce the formulation cost, which in turn would be economically beneficial for the consumers and potentially safer for them as they would be exposed to lesser quantities of dye precursors.

Furthermore, object of the present disclosure is to provide said composition in different cosmetically accepted forms including cream, shampoo, lotion, gel, foam, paste, and mousse.

### SUMMARY

The present disclosure provides a synergistic composition for improving effectiveness of dye precursors to change of colour of hair in a cosmetically acceptable form involving synergistic combination of at least one phosphate ester and at least one carboxylic acid ester at select ratio with significantly reduced quantity of oxidative dye precursors thus advantageously favouring superior colour uptake that can be safer and with a reduced cost. In addition, the composition/kit and the like additionally include other hair benefiting agents.

An aspect of the present disclosure provides, a synergistic a composition for improving effectiveness of dye precursors for change of colour of hair comprising (a) one or more phosphate esters in the ranges of 0.001 to 15 wt %; and (b) one or more carboxylic acid esters in the ranges of 0.001 to 15 wt %; in a ratio ranging from 40:1 to 1:30 co-acting with oxidative dye precursor in combination with oxidising agents in a cosmetically acceptable base.

Various features, aspects and advantages of the inventive subject matter will become more apparent from the following detailed description of preferred embodiments.

### DETAILED DESCRIPTION

It is to be understood that the present disclosure is not limited in its application to the details of construction and the arrangement of components set forth in the following description. The present disclosure is capable of other embodiments, of being practiced, or of being carried out in various ways. Also, it is to be understood that the phraseology and terminology used herein is for the purpose of description and should not be regarded as limiting.

### Definition

All technical and scientific words used herein, unless otherwise defined, have the same meaning as commonly known by one having ordinary ability in the relevant field. The words “a” and “an” designate one or more (i.e., at least one) of the article's grammatical objects. Unless otherwise stated, weight is used to determine all percentages and ratios. All percentages and ratios are calculated based on the total final composition unless otherwise indicated.

As used herein, whether in a transitional phase or the body of a claim, the terms “comprise(s)” and “comprising” are to be interpreted as having an open-ended meaning. That is, the terms are to be interpreted synonymously with the phrases “one or more” or “at least one”. When used in the context of a composition, the term “comprising” means that the process includes at least the recited steps but may include additional steps. When used in the context of a composition, the term “comprising” means that the composition includes at least the recited features or components but may also include additional features or components.

All composition described herein can be performed in suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of the invention otherwise claimed. No language in the specification should be construed as indicating any non-claimed element essential to the practice of the disclosure.

The present disclosure overcomes the aforesaid drawbacks of the prior art and other objects, features, and advantages of the present disclosure will now be described in greater detail. Also, the following description includes various specific details and, is to be regarded as merely exemplary. Accordingly, those of ordinary skill in the art will recognize that without departing from the scope and spirit of the present disclosure and its various embodiments there may be any number of changes and modifications described herein.

The numerical values given for various physical parameters, dimensions and quantities are only approximate values and it is envisaged that the values higher than the numerical value assigned to the physical parameters, dimensions and quantities fall within the scope of the disclosure unless there is a statement in the specification to the contrary.

The term “formulation” used herein refers to a mixture prepared according to a formula. Any other aspect including visual as well as instrumental (spectrophotometric) assessment of colouring are also presented in the tables.
Primary Dye Intermediates (PDIs) used for the invention: PPD=p-Phenylenediamine; p-TDS=Toluene 2,5-diamine sulphate; PAP=p-Aminophenol.
Couplers used: MAP=m-Aminophenol; RL=Resorcinol; DPE=2,4-Diaminophenoxyethanol HCl; AHT=4-Amino 2-hydroxy toluene; 2M5HEAP=2-Methyl-5-hydroxyethylaminophenol.
Concentrations: The tabulated concentrations in the tables are in the form of active percentages and recorded as “weight %” of the total composition.
Colouring Process: In all experiments, unless mentioned otherwise, the colour development was assessed on hair swatches and after application to hair.

The colourant component and the oxidizing components, prepared/packed/stored separately, were combined in a 1:1 ratio or as specified in the relevant tables, mixed well, and applied onto grey hair swatches for which L_{B} values were already determined. Post-application, the hair swatches were left in contact with the mixture. As mentioned in the tables, a colour development time of 10 min or 30 min were provided. After application of colouring mixture as per experimental procedure, the hair swatches were washed first with water followed by a non-conditioning shampoo and finally by water. After colouring, washing, and drying, the L_{A} values were recorded, and the colour uptake (DL) values were calculated.
Evaluations: Colour measurements were undertaken using spectrophotometer and the results were based on the L values. Standard abbreviations for the measurement parameters have been used. Additionally, wherever relevant colours on hair as seen by the eye and visual assessment of extent of colouring (grey hair coverage/colour uptake) has also been documented.
   L_{A}=L value obtained after colouring;
   L_{B}=L value obtained before colouring;
   DL (or L) i.e., Colour Uptake=L_{B}−L_{A}
The effectiveness of the compositions in promoting colour uptake was based on the DL (or L) value. The higher the DL value, the greater is the colour uptake.

An aspect of the present disclosure provides, a synergistic composition for improving effectiveness of dye precursors for change of colour of hair comprising (a) one or more phosphate esters in the ranges of 0.001 to 15 wt %; and (b) one or more carboxylic acid esters in the ranges of 0.001 to 15 wt %; in a ratio ranging from 40:1 to 1:30 co-acting with oxidative dye precursor in combination with oxidising agents in a cosmetically acceptable base.

An embodiment of present disclosure provides, the specific phosphate ester is a combination of Oleth-5 Phosphate and Dioleyl Phosphate.

In an embodiment of present disclosure provides, the carboxylic acid esters consisting of mono/di/tri esters where each of the alcohol part may have 1 to 12 carbons and the acid part may have 1 to 20 carbons exemplary esters being selected from the group consisting of isoamyl formate, isoamyl acetate, isoamyl isovalerate, isoamyl caprylate, isoamyl laurate, isoamyl palmitate, isoamyl myristate, isoamyl stearate, isoamyl oleate, isoamyl cinnamate, isoamyl octanoate, isoamyl behenate, isopropyl palmitate, isopropyl myristate, isopropyl stearate, isopropyl isostearate, isobutyl laurate, vinyl laurate, benzyl laurate, sorbitan monolaurate, isobutyl laurate, oleyl oleate, hexyl laurate, myristyl myristate, cetyl palmitate, 2-butenyl butyrate, 2-ethyl heptenoate, 1-ethenylhexyl butanoate, 2-heptyl butyrate, 2-hexenyl hexanoate, 2-hexenyl isovalerate, allyl butyrate, allyl cyclohexylpropionate, allyl isovalerate, allyl valerate, allyl nonanoate, ascorbyldipalmitate, ascorbyl palmitate, ascorbyl stearate and isoamyl laurate or combinations thereof.

Yet another embodiment of present disclosure provides, the oxidative dye precursors are selected from primary dye intermediates and couplers.

In yet another embodiment of present disclosure provides, the primary dye intermediate selected from the group consisting of p-Phenylenediamine, Toluene 2,5-diamine, N-phenyl p-Phenylenediamine, N,N-bis(2-hydroxyethyl)-p-phenylenediamine, N-ethyl-N-(2-hydroxyethyl)-p-Phenylenediamine, N-ethyl-N-(2-piperidinoethyl)-p-Phenylenediamine, Hydroxypropyl-bis-(N-hydroxyethyl)-p-Phenylenediamine, 2-(1,2-dihydroxyethyl)-p-Phenylenediamine, 2,2′-(2-(4-aminophenylamino)ethylazanediyl)-diethanol, 2-chloro-p-Phenylenediamine, 2-(methoxymethyl) p-Phenylenediamine, p-Aminophenol, p-(methylamino)phenol, 4-Amino-3-methylphenol, 2-Amino-5-ethylphenol, 2,4,5,6-tetraminopyrimidine, 1-hydroxyethyl-4,5-diaminopyrazole sulfate, 4,5-diamino-1-methylpyrazole, 4,5-diamino-1-ethylpyrazole, 4,5-diamino-1-hexylpyrazole and 2-[(3-aminopyrazolo[1,5-a]pyridin-2yl)oxy]ethanol hydrochloride or combination thereof.

Further embodiment of present disclosure provides, the couplers are selected from the group of m-Aminophenol, 2,4-Diaminotoluene, 4-Amino, 2-hydroxytoluene, 3,4-methylenedioxyphenol, 3,4-methylenedioxy-1-(betahydroxyethyl) aminobenzene, 1-methoxy-2-amino-4-(beta-hydroxyethyl)aminobenzene, 1-hydroxy-3-(dimethylamino)benzene, 6-methyl-1-hydroxy-3 (beta-hydroxyethyl) aminobenzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-(beta-hydroxyethyl)aminobenzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3diamino benzene, 1-hydroxy-2-isopropyl-5-methylbenzene, 1,3-dihydroxy benzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methylphenol, 4-hydroxyindole, 4-hydroxyindoline, 6-hydroxyindole and 6-hydroxyindoline, or combination thereof.

In a further embodiment of present disclosure provides, the cosmetically acceptable base selected from alkalizers and developer/oxidant.

Furthermore, embodiment of present disclosure provides, the alkalizers are selected from the group consisting of phosphoric acid, ascorbic acid and lactic acid, or alkalis such as monoethanolamine, diethanolamine, triethanolamine, 2-amino-2-methyl-1-propanol, ammonia, soda lye, potash lye, and tris(hydroxymethyl)-aminomethane or combinations thereof, wherein the pH of the composition is in the ranges of 7 to 11.

Certain embodiment of present disclosure provides, the developer/oxidant is selected from hydrogen peroxide, adducts of hydrogen peroxide, inorganic alkali metal peroxides including barium peroxide, sodium peroxide and inorganic perhydrate salts including the alkali metal salts of perborates, percarbonates, peracids and its salts, persulphates and combinations thereof and more preferably includes hydrogen peroxide, persulphates or combinations thereof.

An embodiment of present disclosure provides, the hair colouration is achieved in less than 45 min.

In an embodiment of present disclosure provides, the conditioning agents are selected from the group consisting of waxes, oils, stearalkoniumchloride, dicetyldimoniumchloride, stearamidopropyldimethylamine, and other quaternary organic compounds or combinations thereof.

In an embodiment of present disclosure provides, the rheology modifiers are selected from the group consisting of acrylate/C10-C30 alkyl acrylate copolymers, cellulose based polymers and gums, homopolymers, copolymers of acrylic or methacrylic acids and their salts polyacrylates, polymethacrylates, polyacrylamides, copolymers of acrylic acid and of acrylamide of sodium salt, sodium salts of polyhydroxycarboxylicacids, crosslinked and/or neutralized 2-acrylamido-2-methylpropanesulphonic acid polymers and copolymers, crosslinked anionic copolymers of acrylamide, acrylates/beheneth-25 methacrylate copolymers, non-ionic guar gums, gums derived from plant exudates or combinations thereof.

In an embodiment of present disclosure provides, a method of colouring the hair involving hair colouring composition/kit involving applying selectively from any of the cream/shampoo/lotion/gel/foam and the like form of hair colouring composition comprising said co-acting at least one phosphate ester in combination with at least one carboxylic acid ester at select ratio along with primary dye intermediates, couplers and in presence of alkalizers and developer/oxidizer on the hair for colouring.

A further aspect of the present disclosure is to provide the composition in different cosmetically accepted forms including cream, shampoo, lotion, gel, foam, paste, and mousse.

Another aspect of the present advancement is providing a method of synergistic composition for promoting uptake of hair colour in hair, involving the steps of:
a) providing said oxidation hair dye formulation in the form of cream/shampoo/lotion/gel/foam/paste/mousse and the like involving alkalizers;
b) providing said developer/oxidiser formulation in the form of cream/shampoo/lotion/gel/foam/paste/mousse and the like;
c) intimately mixing said oxidation hair dye formulation with the developer/oxidant solution wherein the developer/oxidant is provided separately or as a compartmentalized container including sachet in the kit to provide for a ready to use oxidative hair colouring composition mix; and
d) applying the mixture uniformly on dry clean grey hair swatches and then waiting for up to 45 minutes as developmental time.
e) After the specified time, the hair swatches are washed with water followed by shampooing with non-conditioning shampoo, followed by water till no colour is leaching out in the wash liquor;
f) The hair swatches are then dried.

The nature of the present advancement is further illustrated by way of the following non-limiting examples:

### EXAMPLES

As stated hereinbefore, the hair colour uptake-promoting composition/kit comprises at least two components: (a) the base composition involving colourant/oxidation hair dye formulation and (b) the developer/oxidant solution.

All concentrations of ingredients are in weight % of total composition. For dye precursors such as DPE or p-TDS that are present in salt form, the concentrations mentioned represent active components and not the concentrations of their salts.

### Experimental: General Formulations for Colourant

The base compositions used for the colourant incorporating the inventive composition is given in Table 1.

TABLE 1
General Base Formulation for Colourants

| | | Concentrations (weight %) | |
|---|---|---|---|
| | Ingredients | Formula # 1 | Formula # 2 |
| Oil Phase | Cetearyl alcohol | 8.75 | 8.75 |
| | Lauryl alcohol | 0.80 | 0.80 |
| | Steareth-21 | 0.45 | 0.45 |
| | Stearic acid | 0.30 | — |
| | Carboxylic acid esters | As per the relevant experiments | |
| | | (refer to the tables) | |
| | Phosphate ester | As per the relevant experiments | |
| | | (refer to the tables) | |
| Water | Sodium laureth sulphate | 16.00 | 16.00 |
| phase | Cocamidopropyl betaine | 10.00 | 10.00 |
| | Acrylates/Beheneth-25 | 0.50 | 0.50 |
| | Methacrylate Copolymer | | |
| | (Chromapol 5) | | |
| Salt-phase | Sodium sulphite | — | 0.20 |
| | Ascorbic acid | — | 0.20 |
| Dye phase | Monoethanolamine | 3.00 | 3.00 |
| | Primary dye | As per the relevant experiments | |
| | intermediates | (refer to the tables) | |
| | and couplers | | |
| Conditioning | Merquat 2003PR | 1.00 | 1.00 |
| phase & | (Polyquaternium -53) | | |
| Others | Fragrance | 0.30 | 0.30 |
| | Water | QS to 100 | QS to 100 |

### Experimental: General Formulation for Oxidant

The composition of the oxidant is given in Table 2.

TABLE 2
General Formulation for the Oxidant

| | | Concentrations |
|---|---|---|
| | Ingredients | (weight %) |
| Oil Phase | Cetearyl alcohol | 2.50 |
| | Lauryl alcohol | 0.63 |
| Water | Sodium laureth sulphate | 2 |
| phase | Etidronic acid | 1.5 |
| | 2,6-Dicarboxypyridine | 0.1 |
| | Monoethanolamine | 0.33 |
| | Hydrogen peroxide | 18.00 |
| | Water | QS to 100 |

### Example-1: Preparation of Base Formulation for Colourant-Formula #1

1. Oil phase ingredients such as cetearyl alcohol, steareth-21, stearic acid, lauryl alcohol, carboxylic acid esters and phosphate esters (Table-1/Formula #1) were heated to a temperature of 75° C.
2. To the above melt at 75° C., sodium laureth sulfate, cocamidopropyl betaine, and water (5-10%) were added and stirred for 10 min to form an emulsion.
3. Acrylates/Beheneth-25 Methacrylate Copolymer, sold under the trade name Chromapol 5, was separately dispersed in water (5-10%), and added to the main mixture (Step 2).
4. The dye precursors were dispersed in water (15-20%)-monoethanolamine mixture and subsequently added to the emulsion.
5. The mixture was then agitated and cooled to 55° C.
6. Merquat 2003PR (INCI name: polyquaternium 53), fragrance and the remaining quantity of water were added and agitated for a further period of 5 min. It was then cooled to 30° C.

This base formulation was employed for initial data collection with oxidant/developer systems.

For studies with phosphate esters and additional carboxylic acid esters as well as also for other colour shades, a modified base formulation for colourant (Formula #2) was used, which is also given in Table 1.

### Example-2: Preparation of Base Formulation for Colourant-Formula #2

1. Oil phase ingredients such as cetearyl alcohol, steareth-21, lauryl alcohol, phosphate ester and carboxylic acid esters (as per Table-1/Formula #2) are heated to a temperature of 75° C.
2. When the oil phase ingredients are completely melted, the water phase ingredients, mixed with water (5-10%), are added to the mixture at 75° C. and stirred for 10 min to form an emulsion.
3. The salt phase and the dye intermediates are dissolved in water (15-20%)-monoethanolamine mixture and subsequently added to the emulsion.
4. The mixture is then agitated and cooled to 55° C., the conditioning phase (polyquaternium-53) and remaining ingredients (Fragrance and remaining water) are added and agitated for a further period of 5 min. It is then cooled to 30° C.

### Example-3: Preparation of Oxidant

1. Oil phase ingredients are heated to a temperature of 75° C.
2. When the oil phase ingredients are completely melted, sodium laureth sulphate and Etidronic acid along with water (5-10%) were added to the mixture at 75° C. and stirred for 10 min to form the emulsion.
3. Once the mixture cools down to 55° C., a solution of 2,6-Dicarboxypyridine in water (10-15%)-monoethanolamine was added.
4. Hydrogen peroxide and remaining water were added after the emulsion was cooled below 35° C. It was mixed well and was ready for packaging/storing/further use.

### Example-4: Preparation of Hair Swatches

For the preparation of grey hair tresses/swatches, the hair was washed prior to use with non-conditioning shampoo and then dried. Hair swatches—each weighing 5 g—were prepared for the experiments. The tresses were sorted based on the L values before colouring. L values of the swatches selected for the study were 55.5±5.

### Example 5: Process of Colouring of Hair/Hair Swatches and Evaluation

It is recommended to use hand gloves to cover the hands to prevent staining of hands as well as to prevent exposure to the chemicals.

### General Method

The prepared formulations (all the formats) were mixed with developer/oxidant mixture in 1:0.2 to 1:3, preferably in the ratio 1:1 to 1:2. For all the experiments reported in this application, mixing ratio of colourant to developer was 1:1. The mixture was applied on the grey hair swatches with the help of an applicator brush. After application of the mixture, the grey hair swatches were given a 10 or 30 min of development time (as per protocol/relevant Tables). These were then washed with water followed by a non-conditioning shampoo further followed by wash with water and then dried.

A detailed method of colouring the hair involving hair colour uptake promoting composition comprised the steps of:
1. Preparing the oxidation hair dye formulation including the co-acting esters along with primary dye intermediate and coupler system in the desirable form (e.g. cream/shampoo/lotion/gel/foam/paste/mousse and the like) at the desired pH by using pH modifiers;
2. Intimately mixing the said oxidation hair dye formulation with the developer/oxidant solution in the ratio as mentioned in the general method section when the developer/oxidant is provided separately or as a compartmentalized containers including sachet in the kit to provide for a ready to use oxidative hair colouring composition mix; and
3. Applying the said mixture on hair/hair swatches ensuring complete coverage of the hair especially the roots following the guidelines of the general method and stored for the required development time (e.g., 5 or 10 or 30 mins); and
4. Washing first with water followed by a non-conditioning shampoo and further followed by water (under flowing water or shower) and finally drying.

### Measurement of Colour-Uptake:

The La, Lb values were recorded using Colour-Eye 2180 Spectrophotometer (Make: Konica-Minolta). Six (6) measurements per hair swatches were made under D65 illuminant and the average value was recorded. The measurements were made on top, middle and bottom on each side of the hair swatches.

### Observations and Discussion

Generally, in order to achieve 100% grey hair coverage with permanent hair colouring compositions, where the developmental time is 30 min or less than 30 min, the dye concentrations have to be increased.

Typically, the primary dye intermediate concentration in the colourant will be 3.5 to 4% for darker shades such as dark brown and natural black. The dye intermediate concentrations used in a commercial hair colouring product and consequent grey hair coverage are shown in Table 3. PPD concentration in the formulation is 4%. If the concentrations of dye precursors were reduced, say by 50%, the colour uptake is very poor.

TABLE 3
Dependence of Colour Uptake on Concentration of Dye Precursors

| | Colourant Formulation Code |
|---|---|
| | Formula # 2 |
| | Development Time (min) |
| | 10 min |
| Key Ingredients | Concentrations (weight %) |

| Carboxylic acid ester | | — | — |
|---|---|---|---|
| Phosphate ester | | — | — |
| Dye | PPD | 4.0 | 2.0 |
| Precursors | DPE | 0.8 | 0.7 |
| | RL | 1.7 | 0.5 |
| | MAP | 1.5 | 0.7 |

Colour Uptake

| | | |
|---|---|---|
| L_{B} | 50.4 | 52.4 |
| L_{A} | 16.0 | 30.8 |
| DL = (L_{B} − L_{A}) | 34.4 | 21.6 |
| Grey Hair Coverage (Visual) | 100% | Poor |

Thus, it may be seen that with 2% PPD, the DL value was not as high as that obtained with 4% PPD and the visual colour uptake was poor. However, a lower percentage of PPD is always desirable for cost reduction and for lower exposure of consumers to these chemicals that can cause skin allergy.

For the purpose of assessing improvement in colour uptake, at a PPD concentration of 2%, any combination of additional ingredients giving a DL value higher than 21.6 was considered giving an improved in colour uptake.

Among the additional ingredients, role of carboxylic acid esters as well as phosphate esters were studied. The phosphate ester used was a commercially available ingredient named Crodafos HCE (INCI name: Oleth-5 Phosphate (and) Dioleyl Phosphate). The carboxylic acid esters were selected from the esters shown in Table 4. Extensive studies were carried out with these carboxylic acid esters (Samples #1-#12) using Formula #2 for the colourant and using a development time of 10 or 30 mins. These are mentioned in the relevant Tables.

TABLE 4
Sample Codes and Structures of Carboxylic Acid Esters
Used In the Examples

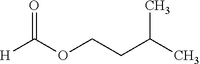

Sample #1
(Isoamyl formate)

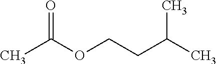

Sample #2
(Isoamyl acetate)

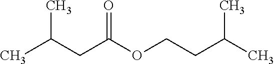

Sample #3
(Isoamyl isovalerate)

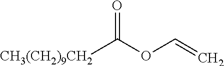

Sample# 4
(Vinyl laurate)

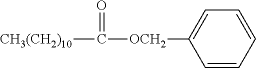

Sample #5
(Benzyl laurate)

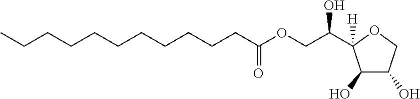 [SMILES: CCCCCCCCCCCC(=O)OC[C@@H](O)[C@H]1OC[C@H](O)[C@H]1O]

Sample #6
(Sorbitan monolaurate)

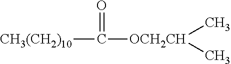

Sample #7
(Isobutyl laurate)

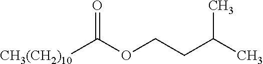

Sample #8
(Isoamyl laurate)

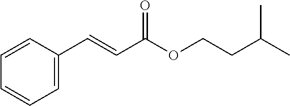 [SMILES: CC(C)CCOC(=O)/C=C/c1ccccc1]

Sample #9
(Isoamyl cinnamate)

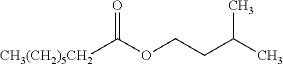

Sample #10
(Isoamyl octanoate)

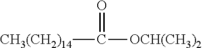

Sample #11
(Isopropyl palmitate)

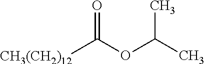

Sample #12
(Isopropyl myristate)

To study the impact on colour uptake of an added carboxylic acid ester or a phosphate ester, these were incorporated into the formulations with reduced quantities of PPD (2%). The concentrations were varied for better understanding of the impact. The results are shown in Table 5.

TABLE 5
Impact of Concentration of Carboxylic Acid Esters or Phosphate Ester
on the Colour Uptake.

| Experiment Numbers | | 1 | 2 | 3 | 4 | 5 | 6 | 7 |
|---|---|---|---|---|---|---|---|---|
| Development Time | | 10 min | | | | | | |
| Key ingredients ( Formula # 2) | | Concentrations (weight % ) | | | | | | |
| Carboxylic acid ester | Isoamyl laurate | 0.5 | 1 | 2 | 4 | — | — | — |
| Phosphate ester | Crodafos HCE | — | — | — | — | 1 | 2 | 4 |
| Dye Precursors | PPD | 2 | 2 | 2 | 2 | 2 | 2 | 2 |
| | DPE | 0.7 | 0.7 | 0.7 | 0.7 | 0.7 | 0.7 | 0.7 |
| | RL | 0.5 | 0.5 | 0.5 | 0.5 | 0.5 | 0.5 | 0.5 |
| | MAP | 0.7 | 0.7 | 0.7 | 0.7 | 0.7 | 0.7 | 0.7 |
| L_{B} | | 53.2 | 52.9 | 53.4 | 52.1 | 52 | 52.9 | 53.6 |
| L_{A} | | 23.8 | 14.8 | 21.8 | 22.9 | 24.6 | 21.8 | 14.3 |
| DL | | 29.4 | 38.2 | 31.6 | 29.2 | 27.4 | 31.1 | 39 |

Table 5 shows the impact of carboxylic acid ester or phosphate ester concentrations on the colour uptake. It may be noted that with the addition of both types of esters individually, very good colour uptake could be achieved which was not possible in their absence (see Table 3). Colour uptake was found to be the best with 1% of isoamyl laurate as well as with 4% of Crodafos HCE (INCI name: Oleth-5 Phosphate (and) Dioleyl Phosphate).

Further, when both a carboxylic acid ester like isoamyl laurate and a phosphate ester, like Crodafos HCE were incorporated in the composition, it was serendipitously observed that it worked synergistically with the dye precursors and delivered improved colour uptake. The results are shown in Table 6.

TABLE 6
Effect Of Combination of Carboxylic Acid Esters with Phosphate
Esters

| Experiment Numbers | | 8 | 9 | 10 | 11 | 12 |
|---|---|---|---|---|---|---|
| Development Time | | 10 min | | | | |
| Key ingredients ( Formula # 2) | | Concentrations (weight % ) | | | | |
| Carboxylic acid ester | Isoamyl | 0 | 0.2 | 0.4 | 0.5 | 0.6 |
| | laurate | | | | | |
| Phosphate ester | Crodafos HCE | 0 | 0.2 | 0.4 | 0.5 | 0.6 |
| Dye Precursors | PPD | 2 | 2 | 2 | 2 | 2 |
| | DPE | 0.7 | 0.7 | 0.7 | 0.7 | 0.7 |
| | RL | 0.5 | 0.5 | 0.5 | 0.5 | 0.5 |
| | MAP | 0.7 | 0.7 | 0.7 | 0.7 | 0.7 |
| L_{B} | | 52.4 | 53.8 | 53.2 | 53.8 | 53.1 |
| L_{A} | | 30.8 | 17.1 | 14.3 | 14.6 | 13.8 |
| DL | | 21.6 | 36.7 | 38.9 | 39.2 | 39.3 |

When both the esters were not used (Expt #8), the DL value was low (21.6) indicating poor colour uptake by hair. This set the benchmark of colour up-take; all DL values that were higher in the presence of the esters were considered to have improvement in colour uptake.

Further, when both the esters were present together, in spite of their lower concentrations, excellent colour uptake was observed. Table 6 shows the impact of combination of the key ingredients (carboxylic acid ester and phosphate ester) on the colour uptake for natural black shade. Without any of these key actives, the DL value was 21.6, indicative of poor colour uptake and coverage (Expt No: 8). In contrast to the high values needed with single ester ingredients (refer Table 5), when these key ingredients were used in combination, surprisingly even at lower concentrations of isoamyl laurate as well as of Crodafos HCE, excellent colour uptake was observed, and it provided improved grey hair coverage.

In order to prove whether any synergy exists between these ingredients, synergy indexes (SI) were calculated from examples given in Table 6.

### Calculation of Synergy:

Synergy was determined by calculating the synergy index ratio following commonly used and accepted method described by Kull A. C, Eisman, P. C, Sylwestrowicz, H. D. and Mayer R. L. Applied microbiology, 9:538-541, 1961.

The synergy index of the composition is calculated as

$$Synergy Index ( SI ) = {Q}_{a} / {Q}_{A} + {Q}_{b} / {Q}_{B}$$

### Where,

Q_{A}—Concentration of the compound A alone which produced the end result;
Qₐ—Concentration of the compound A in the mixture which produced the end result;
Q_{B}—Concentration of the compound B alone which produced the end result;
Q_{b}—Concentration of the compound B in the mixture, which produced the end result.

The result of the interaction can be considered synergistic, additive, or antagonistic depending on the Synergy Index (SI) value. If the SI was <1, the interaction was considered Synergistic, if the SI was >1 the interaction was considered Antagonistic and if the SI was 1, the interaction was Additive.

Using the above method and based on the results presented in Tables 5 and 6, the Synergy Indexes (SI) were calculated at different end points. It can be seen that all the combinations showed synergy (see Table 7).

In the case of isoamyl laurate and Crodafos HCE combinations, the SI was found to be less than 1 in all the cases, proving synergy. Further, the colour uptake values indicate 100% grey hair coverage. The efficacy of isoamyl laurate was

TABLE 7
Calculation Of Synergy Index (SI) And Determination of Synergy

| A = Crodafos HCE; | | | | | | Synergy |
|---|---|---|---|---|---|---|
| B = Isoamyl laurate) | | | | | | Index |
| Colourant composition | DL | Qₐ | Q_{A}* | Q_{b}* | Q_{B}* | (SI ) |
| 4% Crodafos in colourant | 39 | | 2 | | | |
| 1% Isoamyl laurate in | 38.2 | | | | 0.5 | |
| colourant | | | | | | |
| 0.2 IAL + 0.2 Crodafos HCE | 36.7 | 0.1 | 2 | 0.1 | 0.5 | 0.25 |
| 0.4 IAL + 0.4 Crodafos HCE | 38.9 | 0.2 | 2 | 0.2 | 0.5 | 0.5 |
| 0.5 IAL + 0.5 Crodafos HCE | 39.2 | 0.25 | 2 | 0.25 | 0.5 | 0.625 |
| 0.6 IAL + 0.6 Crodafos HCE | 39.3 | 0.3 | 2 | 0.3 | 0.5 | 0.75 |

* Note:
Qₐ, Q_{A}, Q_{b} and Q_{B} concentrations are 50% of the colourant compositions the final mixture for application on hair wash after 1:1 mixing of the colourant to the developer.

improved by 3 to 10 folds when used in combination with Crodafos HCE, whereas 5 efficacy of Crodafos HCE was improved by 6 to 20 folds when used in combination with isoamyl laurate.

To study the effect of ratio between carboxylic acid ester and phosphate ester on the colour uptake different compositions varying the ratio between phosphate ester and isoamyl laurate were prepared (Example 13, 14, 15 and 16). The ratios of Crodafos HCE to isoamyl laurate were varied between 10:1 to 0.1:1. Following the same Hair Colouring Process (Example 5), the formulations were evaluated (Table 8).

TABLE 8
Effect Of Ratio Between Carboxylic
Acid Ester and Phosphate Ester

| | | Experiment No. | | | |
|---|---|---|---|---|---|
| | | 13 | 14 | 15 | 16 |
| | | Development Time | | | |
| | | 10 min | | | |
| Key ingredients (Formula # 2) | | Concentrations (weight %) | | | |
| Carboxylic acid ester | Isoamyl laurate | 0.2 | 0.2 | 0.2 | 0.2 |
| Phosphate ester | Crodafos HCE | 2 | 1 | 0.1 | 0.02 |
| Dye Precursors | PPD | 2 | 2 | 2 | 2 |
| | DPE | 0.7 | 0.7 | 0.7 | 0.7 |
| | RL | 0.5 | 0.5 | 0.5 | 0.5 |
| | MAP | 0.7 | 0.7 | 0.7 | 0.7 |

| | | | | |
|---|---|---|---|---|
| Ratio (Crodafos HCE to Isoamyl laurate) | 10:1 | 5:1 | 0.5:1 | 0.1:1 |

Colour Values (Instrumental Measurements) and Colour Uptake

| | | | | |
|---|---|---|---|---|
| L_{B} | 53.7 | 53.6 | 53.5 | 53.1 |
| L_{A} | 15.6 | 16.1 | 23.2 | 24.4 |
| DL | 38.1 | 37.5 | 30.3 | 28.7 |

It can be seen from Table-8, that while some ratios of phosphate esters (Crodafos HCE) to carboxylic acid esters (isoamyl laurate), such as 10:1 or 5:1, were more effective than the others, the DL values were high (implying good colour uptake) for all concentrations. Based on these results it may be anticipated that specific ratios of select phosphate esters to carboxylic acid esters can be even 40:1 as well as 0.01:1 and can still give excellent colour uptake to hair.

To establish the colour up-take effect at low concentrations of phosphate ester and carboxylic acid esters disclosed in Table-4 individually, concentrations such as 0.0025%, 0.005% and 0.01% were used in the formulations and they were evaluated with the oxidant formulation in 1:1 ratio for 10 min developmental time. The results are tabulated in Tables 9a-c.

TABLE 9a
Effect of Low Concentrations of Phosphate Esters and Carboxylic Acid Esters

| Colourant Formulation: | | Phosphate Ester | | | | | | |
|---|---|---|---|---|---|---|---|---|
| Formula # 2 | | (Crodafos HCE) | | | Sample 1 | | Sample 2 | |
| | | Concentrations (weight % ) | | | | | | |
| Carboxylic acid ester | | — | — | — | 0.005 | 0.01 | 0.005 | 0.01 |
| Phosphate ester | | 0.0025 | 0.005 | 0.01 | — | — | — | — |
| Development Time | | 10 min | | | | | | |
| Dye Precursors | PPD | 2 | 2 | 2 | 2 | 2 | 2 | 2 |
| | DPE | 0.7 | 0.7 | 0.7 | 0.7 | 0.7 | 0.7 | 0.7 |
| | RL | 0.5 | 0.5 | 0.5 | 0.5 | 0.5 | 0.5 | 0.5 |
| | MAP | 0.7 | 0.7 | 0.7 | 0.7 | 0.7 | 0.7 | 0.7 |

| | | | | | | | |
|---|---|---|---|---|---|---|---|
| Ratio of phosphate ester | NA | NA | NA | NA | NA | NA | NA |
| to carboxylic acid ester | | | | | | | |

Colour Values ( Instrumental Measurements) and Colour Uptake

| | | | | | | | |
|---|---|---|---|---|---|---|---|
| L_{B} | 54.7 | 53 | 53.14 | 54.57 | 54.02 | 54.73 | 52.66 |
| L_{A} | 38.97 | 30.21 | 31.39 | 36.73 | 39.54 | 37.65 | 34.68 |
| DL | 15.73 | 22.79 | 21.75 | 17.84 | 14.48 | 17.08 | 17.98 |

(Note:
NA = Not Applicable)

TABLE 9b
Effect of Low Concentrations of Phosphate Esters And Carboxylic Acid Esters

| Colourant Formulation: | | | | | | | | | | | |
|---|---|---|---|---|---|---|---|---|---|---|---|
| Formula # 2 | | Sample 3 | | Sample 4 | | Sample 5 | | Sample 6 | | Sample 7 | |
| | | Concentrations (weight % ) | | | | | | | | | |
| Carboxylic acid ester | | 0.005 | 0.01 | 0.005 | 0.01 | 0.005 | 0.01 | 0.005 | 0.01 | 0.005 | 0.01 |
| Phosphate ester (Crodafos HCE) | | — | — | — | — | — | — | — | — | — | — |
| Development Time | | 10 min | | | | | | | | | |
| Dye Precursors | PPD | 2 | 2 | 2 | 2 | 2 | 2 | 2 | 2 | 2 | 2 |
| | DPE | 0.7 | 0.7 | 0.7 | 0.7 | 0.7 | 0.7 | 0.7 | 0.7 | 0.7 | 0.7 |
| | RL | 0.5 | 0.5 | 0.5 | 0.5 | 0.5 | 0.5 | 0.5 | 0.5 | 0.5 | 0.5 |
| | MAP | 0.7 | 0.7 | 0.7 | 0.7 | 0.7 | 0.7 | 0.7 | 0.7 | 0.7 | 0.7 |

| | | | | | | | | | | |
|---|---|---|---|---|---|---|---|---|---|---|
| Ratio of phosphate ester to | NA | NA | NA | NA | NA | NA | NA | NA | NA | NA |
| carboxylic acid ester | | | | | | | | | | |

Colour Values ( Instrumental Measurements) and Colour Uptake

| | | | | | | | | | | |
|---|---|---|---|---|---|---|---|---|---|---|
| L_{B} | 53.18 | 52.82 | 54.35 | 52.54 | 53.21 | 53.94 | 53.53 | 53.43 | 54.59 | 52.9 |
| L_{A} | 30.83 | 38.33 | 36.45 | 32.81 | 32.57 | 33.65 | 31.75 | 34.38 | 35.59 | 31.88 |
| DL | 22.35 | 14.49 | 17.9 | 19.73 | 20.64 | 20.29 | 21.78 | 19.05 | 19 | 21.02 |

(Note:
NA = Not Applicable)

TABLE 9c
Effect of Low Concentrations of Phosphate Esters and Carboxylic Acid Esters

| Colourant Formulation : | | | | | | | | | | | |
|---|---|---|---|---|---|---|---|---|---|---|---|
| Formula # 2 | | Sample 8 | | Sample 9 | | Sample 10 | | Sample 11 | | Sample 12 | |
| | | Concentrations (weight % ) | | | | | | | | | |
| Carboxylic acid ester | | 0.005 | 0.01 | 0.005 | 0.01 | 0.005 | 0.01 | 0.005 | 0.01 | 0.005 | 0.01 |
| Phosphate ester | | — | — | — | — | — | — | — | — | — | — |
| (Crodafos HCE) | | | | | | | | | | | |
| Development Time | | 10 min | | | | | | | | | |
| Dye Precursors | PPD | 2 | 2 | 2 | 2 | 2 | 2 | 2 | 2 | 2 | 2 |
| | DPE | 0.7 | 0.7 | 0.7 | 0.7 | 0.7 | 0.7 | 0.7 | 0.7 | 0.7 | 0.7 |
| | RL | 0.5 | 0.5 | 0.5 | 0.5 | 0.5 | 0.5 | 0.5 | 0.5 | 0.5 | 0.5 |
| | MAP | 0.7 | 0.7 | 0.7 | 0.7 | 0.7 | 0.7 | 0.7 | 0.7 | 0.7 | 0.7 |

| | | | | | | | | | | |
|---|---|---|---|---|---|---|---|---|---|---|
| Ratio of phosphate ester | NA | NA | NA | NA | NA | NA | NA | NA | NA | NA |
| to carboxylic acid ester | | | | | | | | | | |

Colour Values (Instrumental Measurements) and Colour Uptake

| | | | | | | | | | | |
|---|---|---|---|---|---|---|---|---|---|---|
| L_{B} | 54.94 | 52.1 | 53.68 | 53.78 | 54.76 | 52.92 | 54.23 | 53.05 | 53.23 | 53.24 |
| L_{A} | 33.77 | 31.76 | 33.97 | 36 | 34.53 | 34.32 | 30.22 | 32.6 | 32.7 | 25.9 |
| DL | 21.17 | 20.34 | 19.71 | 17.78 | 20.23 | 18.6 | 24.01 | 20.45 | 20.53 | 27.34 |

(Note:
NA = Not Applicable)

From the Tables 9a-c, it was evident, that when either the phosphate ester or the carboxylic acid esters (Table-4) were used individually at lower concentrations, the colour uptake was found to be poor.

But when the phosphate ester and the carboxylic acid esters were combined, the colour uptake was found to improve significantly indicating synergistic action of combination of phosphate esters and carboxylic acid esters (Table 10a-b).

TABLE 10a
Effect of Combinations of Phosphate Esters and Carboxylic Acid Esters

| | | Sample 1 | Sample 2 | Sample 3 | Sample 4 | Sample 5 | Sample 6 |
|---|---|---|---|---|---|---|---|
| Colourant Formulation | | + | + | + | + | + | + |
| (Formula # 2) | | Phosphate | Phosphate | Phosphate | Phosphate | Phosphate | Phosphate |
| | | Concentrations (weight % ) | | | | | |
| Carboxylic acid ester | | 0.005 | 0.005 | 0.005 | 0.005 | 0.005 | 0.005 |
| Phosphate ester (Crodafos HCE) | | 0.005 | 0.005 | 0.005 | 0.005 | 0.005 | 0.005 |
| Development Time (min) | | 10 min | | | | | |
| Dye Precursors | PPD | 2 | 2 | 2 | 2 | 2 | 2 |
| | DPE | 0.7 | 0.7 | 0.7 | 0.7 | 0.7 | 0.7 |
| | RL | 0.5 | 0.5 | 0.5 | 0.5 | 0.5 | 0.5 |
| | MAP | 0.7 | 0.7 | 0.7 | 0.7 | 0.7 | 0.7 |

| | | | | | | |
|---|---|---|---|---|---|---|
| Ratio of phosphate ester to | 1:1 | 1:1 | 1:1 | 1:1 | 1:1 | 1:1 |
| carboxylic acid ester | | | | | | |

Colour Values (Instrumental Measurements) and Colour Uptake

| | | | | | | |
|---|---|---|---|---|---|---|
| L_{B} | 53.71 | 53.49 | 54.69 | 55.01 | 54.99 | 55.43 |
| L_{A} | 29.32 | 26.98 | 32.88 | 27.03 | 28.3 | 27.67 |
| DL | 24.39 | 26.51 | 21.81 | 27.98 | 26.69 | 27.76 |

TABLE 10b
Effect of Combinations of Phosphate Esters and Carboxylic Acid Esters

| | | Sample 7 | Sample 8 | Sample 9 | Sample 10 | Sample 11 | Sample 12 |
|---|---|---|---|---|---|---|---|
| Colourant Formulation | | + | + | + | + | + | + |
| (Formula # 2) | | Phosphate | Phosphate | Phosphate | Phosphate | Phosphate | Phosphate |
| | | Concentrations (weight % ) | | | | | |
| Carboxylic acid ester | | 0.005 | 0.005 | 0.005 | 0.005 | 0.005 | 0.005 |
| Phosphate ester | | 0.005 | 0.005 | 0.005 | 0.005 | 0.005 | 0.005 |
| (Crodafos HCE) | | | | | | | |
| Development Time (min) | | 10 min | | | | | |
| Dye Precursors | PPD | 2 | 2 | 2 | 2 | 2 | 2 |
| | DPE | 0.7 | 0.7 | 0.7 | 0.7 | 0.7 | 0.7 |
| | RL | 0.5 | 0.5 | 0.5 | 0.5 | 0.5 | 0.5 |
| | MAP | 0.7 | 0.7 | 0.7 | 0.7 | 0.7 | 0.7 |

| | | | | | | |
|---|---|---|---|---|---|---|
| Ratio of phosphate ester | 1:1 | 1:1 | 1:1 | 1:1 | 1:1 | 1:1 |
| to carboxylic acid ester | | | | | | |

Colour Values ( Instrumental Measurements) and Colour Uptake

| | | | | | | |
|---|---|---|---|---|---|---|
| L_{B} | 55.14 | 55.07 | 55.47 | 54.51 | 55.03 | 55.43 |
| L_{A} | 24.2 | 25.71 | 25.63 | 30.77 | 23.55 | 27.67 |
| DL | 30.94 | 29.36 | 29.84 | 23.74 | 31.48 | 27.76 |

As a representative example, when 0.01% phosphate ester was used alone (Table 9a) DL was 21.75 and when 0.01% of Sample 4 was used alone (Table 9b), the DL value was 19.73. But, when these two are used in combination at a concentration of 0.005% each (refer Table 10a), and thus the total ester concentration was 0.01%, DL value was found to be 27.98. This clearly indicates that, synergy exists between phosphate ester and carboxylic acid esters and consequently there is an improved colour uptake in hair. The result was found to be consistent for all the carboxylic acid esters (refer Table 10 a-b), further demonstrating the synergistic behaviour of the combination across diverse structures.

In order to establish the effective ratios between carboxylic acid esters and phosphate esters, a series of experiments are performed and the details of which are given in Table 11.

TABLE 11
Establishing the Ratio Between Carboxylic Acid Esters and Phosphate Ester

| | | |
|---|---|---|
| Colourant | | |
| Formulation | | |
| (Formula # 2) | Sample 3 + Phosphate | Sample 6 + Phosphate |

Concentrations (weight % )

| Carboxylic | | 0.05 | 0.05 | 0.05 | 0.05 | 0.2 | 0.05 | 0.05 | 0.05 | 0.05 | 0.2 |
|---|---|---|---|---|---|---|---|---|---|---|---|
| acid ester | | | | | | | | | | | |
| Phosphate ester | | 0.05 | 0.2 | 0.5 | 1 | 0.05 | 0.05 | 0.2 | 0.5 | 1 | 0.05 |
| (Crodafos HCE) | | | | | | | | | | | |
| Development | | | | | | | | | | | |
| Time (min) | | 10 min | | | | | | | | | |
| Dye | PPD | 2 | 2 | 2 | 2 | 2 | 2 | 2 | 2 | 2 | 2 |
| Pre- | DPE | 0.7 | 0.7 | 0.7 | 0.7 | 0.7 | 0.7 | 0.7 | 0.7 | 0.7 | 0.7 |
| cursors | RL | 0.5 | 0.5 | 0.5 | 0.5 | 0.5 | 0.5 | 0.5 | 0.5 | 0.5 | 0.5 |
| | MAP | 0.7 | 0.7 | 0.7 | 0.7 | 0.7 | 0.7 | 0.7 | 0.7 | 0.7 | 0.7 |

| | | | | | | | | | | |
|---|---|---|---|---|---|---|---|---|---|---|
| Ratio of | 1:1 | 4:1 | 10:1 | 20:1 | 1:4 | 1:1 | 4:1 | 10:1 | 20:1 | 1:4 |
| phosphate | | | | | | | | | | |
| ester to | | | | | | | | | | |
| carboxylic | | | | | | | | | | |
| acid ester | | | | | | | | | | |

Colour Values (Instrumental Measurements) and Colour Uptake

| | | | | | | | | | | |
|---|---|---|---|---|---|---|---|---|---|---|
| L_{B} | 53.2 | 53.83 | 53.42 | 53.69 | 54.2 | 54.22 | 54.39 | 54.13 | 54.19 | 54.11 |
| L_{A} | 19.66 | 31.79 | 22.35 | 21.45 | 20.28 | 19.23 | 22.35 | 19.49 | 17.87 | 21.85 |
| DL | 33.54 | 22.04 | 31.07 | 32.24 | 33.92 | 34.99 | 32.04 | 34.64 | 36.32 | 32.26 |

Sample 9 + Phosphate

| Carboxylic | | 0.05 | 0.05 | 0.05 | 0.05 | 0.2 |
|---|---|---|---|---|---|---|
| acid ester | | | | | | |
| Phosphate ester | | 0.05 | 0.2 | 0.5 | 1 | 0.05 |
| (Crodafos HCE) | | | | | | |
| Development | | | | | | |
| Time (min) | | | | | | |
| Dye | PPD | 2 | 2 | 2 | 2 | 2 |
| Precursors | DPE | 0.7 | 0.7 | 0.7 | 0.7 | 0.7 |
| | RL | 0.5 | 0.5 | 0.5 | 0.5 | 0.5 |
| | MAP | 0.7 | 0.7 | 0.7 | 0.7 | 0.7 |

| | | | | | |
|---|---|---|---|---|---|
| Ratio of phosphate | 1:1 | 4:1 | 10:1 | 20:1 | 1:4 |
| ester to carboxylic | | | | | |
| acid ester | | | | | |

Colour Values (Instrumental Measurements) and Colour Uptake

| | | | | | |
|---|---|---|---|---|---|
| L_{B} | 55.86 | 55.2 | 55.02 | 55.26 | 55.99 |
| L_{A} | 18.9 | 31.2 | 19.95 | 17.69 | 21.38 |
| DL | 36.96 | 24 | 35.07 | 37.57 | 34.61 |

Table 11 shows different ratios of phosphate ester to carboxylic acid ester tried for sample 3, 6 and 9. The ratios tried were 1:1, 4:1, 10:1, 20:1 and 1:4. In all the ratios evaluated (refer Tables 8 and 11), the compositions were found to be effective in enhancing the colour uptake in hair and the DL values were found to have improved significantly compared to the single ingredients alone (refer Tables 9a-c).

In order to establish the co-action of carboxylic acid esters with phosphate esters on reducing the dye precursors, isoamyl octanoate (Sample 10) was used in combination with Crodafos HCE. PPD levels were reduced from 2 wt % to 1 wt % and to 0.5 wt %, at two different concentrations of the esters (0.005 wt % of each

TABLE 12
Reduction In Quantities of Dye Precursors

| Colourant Formulation | | Sample 10 + Phosphate ester | | | |
|---|---|---|---|---|---|
| (Formula # 2) | | Concentrations (weight %) | | | |
| Carboxylicacid ester | | 0.005 | 0.01 | 0.005 | 0.005 |
| Phosphate ester (Crodafos HCE) | | 0.005 | 0.01 | 0.005 | 0.005 |
| Development Time (min) | | 10 mins | | 30 mins | |
| Dye | PPD | 1 | 0.5 | 1 | 0.5 |
| Precursors | DPE | 0.2 | 0.1 | 0.2 | 0.1 |
| | RL | 0.5 | 0.25 | 0.5 | 0.25 |
| | MAP | 0.7 | 0.35 | 0.7 | 0.35 |

| | | | | |
|---|---|---|---|---|
| Total Quantity of Dye | 2.4 | 1.2 | 2.4 | 1.2 |
| Precursors | | | | |
| Ratio of phosphate ester to | 1:1 | 1:1 | 1:1 | 1:1 |
| carboxylic acid ester | | | | |

Colour Values (Instrumental Measurements) and Colour Uptake

| | | | | |
|---|---|---|---|---|
| L_{B} | 56.04 | 56.75 | 56.28 | 56.17 |
| L_{A} | 25.45 | 30.04 | 24.84 | 22.13 |
| DL | 30.59 | 26.71 | 31.44 | 34.04 |

and 0.01 wt % of each) at a 1:1 ratio. The total weight % of the dye precursors were 2.4 wt % and 1.2 wt % respectively compared to the requirement of 3.9 wt % of total dye precursors with 2 wt % of PPD (refer e.g. Table Sep. 10, 2011) and 8 wt % of total dye precursor with 4 wt % of PPD (refer Table 3). For 10 min developmental time, DL values were 30.59 and 26.71, indicating improved colour uptake. Additionally, it was observed that with 30 min developmental time, the DL values were enhanced to 31.44 and 34.04, respectively indicating even better colour uptake.

The observation that a development time of 30 minutes can enhance the colour uptake was further verified with different carboxylic acid esters. Also, this aspect of longer time for development was useful in lowering the amount of total dye precursors. The results are presented in Table 13.

TABLE 13
Effect of Longer Development Time on Uptake of Colour in Hair

| Colourant Formulation: | | Sample 3 + | | Sample 6 + | | | | Sample 7 + | | |
|---|---|---|---|---|---|---|---|---|---|---|
| Formula # 2 | | Phosphate ester | | Phosphate ester | | | | Phosphate ester | | |
| | | Concentrations (weight % ) | | | | | | | | |
| Carboxylic acid ester | | 0.005 | 0.005 | 0.005 | 0.005 | 0.0025 | 0.005 | 0.005 | 0.005 | 0.005 |
| Phosphate ester | | 0.005 | 0.005 | 0.005 | 0.005 | 0.0025 | 0.005 | 0.005 | 0.005 | 0.005 |
| (Crodafos HCE) | | | | | | | | | | |
| Development Time (min) | | 10 mins | 30 mins | 10 mins | 30 mins | | | 10 mins | 30 mins | |
| Dye Precursors | PPD | 1 | 1 | 2 | 2 | 2 | 0.5 | 2 | 2 | 1 |
| | DPE | 0.2 | 0.2 | 0.7 | 0.7 | 0.7 | 0.1 | 0.7 | 0.7 | 0.2 |
| | RL | 0.5 | 0.5 | 0.5 | 0.5 | 0.5 | 0.25 | 0.5 | 0.5 | 0.5 |
| | MAP | 0.7 | 0.7 | 0.7 | 0.7 | 0.7 | 0.35 | 0.7 | 0.7 | 0.7 |

| | | | | | | | | | |
|---|---|---|---|---|---|---|---|---|---|
| Total Quantity of Dye | 2.4 | 2.4 | 3.9 | 3.9 | 3.9 | 1.2 | 3.9 | 3.9 | 2.4 |
| Precursors | | | | | | | | | |
| Ratio of phosphate ester | 1:1 | 1:1 | 1:1 | 1:1 | 1:1 | 1:1 | 1:1 | 1:1 | 1:1 |
| to carboxylic acid ester | | | | | | | | | |

Colour Values (Instrumental Measurements) and Colour Uptake

| | | | | | | | | | |
|---|---|---|---|---|---|---|---|---|---|
| L_{B} | 55.7 | 55.43 | 55.43 | 55.8 | 56.75 | 55.2 | 55.14 | 55.01 | 54.21 |
| L_{A} | 33.27 | 28.76 | 27.67 | 25.57 | 30.04 | 30 | 24.2 | 19.92 | 25.46 |
| DL | 22.43 | 26.67 | 27.76 | 30.23 | 26.71 | 25.2 | 30.94 | 35.09 | 28.75 |

It can be observed that with the same composition or with minor variation of composition, a development time of 30 min resulted in greater value for DL reflecting superior/improved colour uptake. Development time of 30 min was also effective in lowering the total amount of dye precursors and also in reducing the weight percentage of both carboxylic acid ester as well as phosphate esters to 0.0025 wt % each.

While the Formulas #1 and #2 give black colour to hair, it may be anticipated that the synergistic combination of the phosphate ester and carboxylic acid ester can be effective in producing different colours too. Table 14 gives a few non-limiting examples of other colours that could be developed by different dye precursor combinations in presence of the synergistic combination of phosphate ester (Crodafos HCE) and carboxylic acid ester (Sample 9).

TABLE 14
Different Primary Dye Intermediates And
Couplers To Deliver Different Colours

| Colourant Formulation: | | Sample 9 + Phosphate | | Sample 9 + Phosphate | |
|---|---|---|---|---|---|
| Formula# 2 | | Concentrations (weight %) | | | |
| Colour Shade | | Dark Brown | | Burgundy | |
| Development Time (min) | | 10 mins | 30 mins | 10 mins | 30 mins |
| Carboxylic acid ester | | 0.2 | 0.2 | 0.2 | 0.2 |
| Phosphate ester | | 0.2 | 0.2 | 0.2 | 0.2 |
| (Crodafos HCE) | | | | | |
| Dye | PPD | 1.8 | 1.8 | 0.65 | 0.65 |
| precursors | DPE | 0.2 | 0.2 | 0 | 0 |
| | RL | 1 | 1 | 0.1 | 0.1 |
| | MAP | 1.5 | 1.5 | 0 | 0 |
| | PAP | 0.24 | 0.24 | 0 | 0 |
| | AHT | 0 | 0 | 0.5 | 0.5 |
| | 2M5HEAP | 0 | 0 | 0.1 | 0.1 |
| | p-TDS | 0 | 0 | 0.05 | 0.05 |

| | | | | |
|---|---|---|---|---|
| Ratio of phosphate ester | 1:1 | 1:1 | 1:1 | 1:1 |
| to carboxylic acid ester | | | | |

Colour Values (Instrumental Measurements) and Colour Uptake

| | | | | |
|---|---|---|---|---|
| L_{B} | 57.2 | 57.3 | 55.9 | 56.8 |
| L_{A} | 32.65 | 26.12 | 30.2 | 27.23 |
| DL | 24.55 | 31.18 | 25.7 | 29.57 |

As a result, an embodiment of the present disclosure includes illustrations and tables demonstrating a synergistic composition that provides enhanced colour uptake in hair with oxidative hair colourants resulting in uniform grey/blond/bleached hair coverage in desired colour shade within a colour developmental period of less than 45 min.

Thus, the present synergistic composition that enhances colour uptake in hair involving suitable combinations of carboxylic acid esters with specific phosphate esters and dye precursors (primary dye intermediates and couplers) presents a technological advantage that can substantially reduce the requirement of dye precursors in the hair colouring products rendering them safer with reduced risk of allergic reaction as well as more economical.

### ADVANTAGES

1. Cost-effective.
2. The present disclosure provides a safe composition.
3. The present disclosure provides an efficient synergistic composition.
4. The present disclosure provides a composition giving excellent grey hair coverage.

1. A composition for improving effectiveness of dye precursors for change of colour of hair comprising:
(a) one or more phosphate esters in the ranges of 0.001 to 15 wt %; and
(b) one or more carboxylic acid esters in the ranges of 0.001 to 15 wt %;
in a ratio ranging from 40:1 to 1:30 co-acting with oxidative dye precursor in combination with oxidising agents in a cosmetically acceptable base.

2. The composition as claimed in claim 1, wherein the phosphate ester is a combination of Oleth-5 Phosphate and Dioleyl Phosphate.

3. The composition as claimed in claim 1, wherein the carboxylic acid esters consisting of mono/di/tri esters where each of the alcohol part have 1 to 12 carbons and the acid part have 1 to 20 carbons being selected from the group consisting of isoamyl formate, isoamyl acetate, isoamyl isovalerate, isoamyl caprylate, isoamyl laurate, isoamyl palmitate, isoamyl myristate, isoamyl stearate, isoamyl oleate, isoamyl cinnamate, isoamyl octanoate, isoamyl behenate, isopropyl palmitate, isopropyl myristate, isopropyl stearate, isopropyl isostearate, isobutyl laurate, vinyl laurate, benzyl laurate, sorbitan monolaurate, isobutyl laurate, oleyl oleate, hexyl laurate, myristyl myristate, cetyl palmitate, 2-butenyl butyrate, 2-ethyl heptenoate, 1-ethenylhexyl butanoate, 2-heptyl butyrate, 2-hexenylhexanoate, 2-hexenylisovalerate, allylbutyrate, allylcyclohexylpropionate, allylisovalerate, allylvalerate, allylnonanoate, ascorbyldipalmitate, ascorbyl palmitate, ascorbyl stearate and isoamyl laurate or combinations thereof.

4. The composition as claimed in claim 1, wherein the oxidative dye precursors are selected from primary dye intermediates and couplers.

5. The composition as claimed in claim 4, wherein the primary dye intermediate are selected from the group consisting of p-Phenylenediamine, Toluene 2,5-diamine, N-phenyl p-phenylenediamine, N,N-bis(2-hydroxyethyl)-p-phenylenediamine, N-ethyl-N-(2-hydroxyethyl)-p-Phenylenediamine, N-ethyl-N-(2-piperidinoethyl)-p-Phenylenediamine, Hydroxypropyl-bis-(N-hydroxyethyl)-p-Phenylenediamine, 2-(1,2-dihydroxyethyl)-p-Phenylenediamine, 2,2′-(2-(4-aminophenylamino)ethylazanediyl)-diethanol, 2-chloro-p-Phenylenediamine, 2-(methoxymethyl) p-Phenylenediamine, p-Aminophenol, p-(methylamino)phenol, 4-Amino-3-methylphenol, 2-Amino-5-ethylphenol, 2,4,5,6-tetraminopyrimidine, 1-hydroxyethyl-4,5-diaminopyrazole sulfate, 4,5-diamino-1-methylpyrazole, 4,5-diamino-1-ethylpyrazole, 4,5-diamino-1-hexylpyrazole and 2-[(3-aminopyrazolo[1,5-a]pyridin-2yl)oxy]ethanol hydrochloride or combination thereof.

6. The composition as claimed in claim 4, wherein the couplers are selected from the group consisting of m-Aminophenol, 2,4-Diaminotoluene, 4-Amino, 2-hydroxytoluene, 3,4-methylenedioxyphenol, 3,4-methylenedioxy-1-(betahydroxyethyl)aminobenzene, 1-methoxy-2-amino-4-(beta-hydroxyethyl) aminobenzene, 1-hydroxy-3-(dimethylamino)benzene, 6-methyl-1-hydroxy-3 (betahydroxyethyl)aminobenzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-aminobenzene, 6-(beta-methoxy-1-amino-3-(beta-hydroxyethyl) aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diamino benzene, 1-hydroxy-2-isopropyl-5-methylbenzene, 1,3-dihydroxybenzene, 2-chloro-1,3-dihydroxybenzene, 2-methyl-1,3-dihydroxy benzene, 4-chloro-1,3-dihydroxybenzene, 5,6-dichloro-2-methyl-1,3-dihydroxybenzene, 1-hydroxy-3-amino-benzene, 1-hydroxy-(carbamoyl methylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxy naphthalene, 1,7-dihydroxynaphthalene, 1,5-dihydroxynaphthalene, 5-amino-2-methylphenol, 4-hydroxy indole, 4-hydroxyindoline, 6-hydroxyindole and 6-hydroxyindoline, or combination thereof.

7. The composition as claimed in claim 1, wherein the cosmetically acceptable base is selected from alkalizers and developer/oxidant.

8. The composition as claimed in claim 7, wherein the alkalizers are selected from the group consisting of phosphoric acid, ascorbic acid and lactic acid, or alkalis such as monoethanolamine, diethanolamine, triethanolamine, 2-amino-2-methyl-1-propanol, ammonia, soda lye, potash lye, and tris(hydroxymethyl) aminomethane or combinations thereof.

9. The composition as claimed in claim 7, wherein the developer/oxidant is selected from the group consisting of hydrogen peroxide, adducts of hydrogen peroxide, inorganic alkali metal peroxides including barium peroxide, sodium peroxide and inorganic perhydrate salts including the alkali metal salts of perborates, percarbonates, peracids and its salts and persulphates or combinations thereof.
